(19) United States
(12) Patent Application Publication
(10) Pub. No.: US 2008/0057129 A1
(43) Pub. Date: Mar. 6, 2008

(54) Drug microparticles

(76) Inventors: E. Itzhak Lerner, Petach Tikva (IL); Moshe Flashner-Barak, Petach Tikva (IL); Ruud Smit, TJ Haarlem (NL); Richard Van Lamoen, VJ Utrecht (NL); Erwin Van Achthoven, EM Leiderdorp (NL); Hans Keegstra, CR Alkmaar (NL)

Correspondence Address: KENYON & KENYON LLP, ONE BROADWAY, NEW YORK, NY, 10004, US

(21) Appl. No.: 11/732,705

(22) Filed: Apr. 3, 2007

Related U.S. Application Data
(60) Provisional application No. 60/789,197, filed on Apr. 3, 2006.
(60) Provisional application No. 60/854,778, filed on Oct. 26, 2006.

(51) Int. Cl. A61K 9/14; A61K 31/047; A61K 31/133; A61K 31/337; A61K 31/351; A61K 31/435; A61P 11/00; A61K 31/4353; A61K 31/56; A61K 31/58; A61K 31/59; A61K 31/70

(52) U.S. Cl. 424/489; 514/167; 514/174; 514/178; 514/29; 514/291; 514/449; 514/459; 514/534; 514/653; 514/738

(57) ABSTRACT

Pharmaceutical compositions are described containing carrier particles bearing microparticles of a drug. The drug microparticles may be deposited on the carrier particles, for example, by sublimation. Preferred embodiments of these pharmaceutical compositions are suitable for administration by inhalation or injection. Methods for treating lung infection in patients with cystic fibrosis through inhalation of, for example, calcitriol compositions, are also described.

### RELATED APPLICATIONS

This application claims priority from U.S. Provisional Patent Application No. 60/789,197, filed Apr. 3, 2006, and from U.S. Provisional Patent Application No. 60/854,778, filed Oct. 26, 2006, each of which is incorporated herein by reference in its entirety.

### FIELD OF THE INVENTION

The present invention relates to microparticles of drugs, especially drugs that are poorly soluble in water.

### BACKGROUND OF THE INVENTION

Many important drugs have poor oral bioavailability because they are poorly soluble in water. Many approaches have been suggested to overcome this problem. Although some approaches have been used with limited commercial success, each approach has its own drawbacks and limitations.

The bioavailability of poorly water-soluble drugs may be improved by decreasing the particle size of the drug to increase the surface area. Milling, high pressure homogenization, spray drying, lyophilization of solutions in water—organic solvent mixtures, and lyophilization of solutions of inorganic solvents have been tried. Size reduction is, in principal, generally applicable for improving bioavailability, but achieving size reduction by, for example, high energy milling, requires special equipment and is not always applicable. High pressure homogenization requires special equipment and requires organic solvents that can remain in the comminuted product. Spray drying also requires solvents and generally produces larger size particles.

Many of the above-described techniques require forming particles by solvent removal which, in turn, entails concentration of a solution. During solution concentration, solute molecules, which in solution are statistically separated into individual molecules and small clusters or aggregates, are drawn together to form larger molecular aggregates. When the solute drug eventually precipitates, relatively larger crystals are formed.

Lyophilization (freeze drying) has the advantage of allowing the solvent to be removed while keeping the solute relatively immobile, thereby suppressing enlargement of clusters or aggregates. When the solvent is removed, the formed crystals are smaller or the material is amorphous, reflecting the separation of the molecules in the frozen solution state. Molecular separation can be improved and aggregate formation still further suppressed by lyophilizing a more dilute solution, although the energy requirements for removing more solvent may be increased. Lyophilization is usually a very slow, energy intensive process and usually requires high vacuum equipment. Furthermore, there is a tendency for the crystals formed to aggregate in the free state, undoing the job that the freeze drying did. This tendency can sometimes be overcome with additives, but these must be compatible with the entire system.

Amorphous or nanoparticulate materials tend to show poor bulk flow properties as powders, requiring formulation work to be able to fill them into capsules. While these problems are not insurmountable, they add further limitations in the usefulness of the system. Many of the existing limitations are overcome by preferred embodiments of the present invention.

It is sometimes desirable to administer a drug, including a poorly water soluble drug, to a patient (i.e., deliver the drug to the circulatory system or the situs of the disease) through the respiratory system. This can be referred to as inhalation administration or inhalation delivery.

For inhalation administration the size of the particles is reported to be important. See, e.g., Howard C. Ansel, Ph.D. et al., Pharmaceutical Dosage Forms and Drug Delivery Systems, p. 384 (Donna Bolado, ed., 7^{th} ed.)

The particle size distribution of the active pharmaceutical ingredients used in dry powder inhalation (DPI) products is believed to be critical for the aerodynamic performance of the composition being inhaled. Generally, only particles with a size less than 5 μm are effective to penetrate to the desired depth in the lungs. For this reason the active ingredient is commonly milled using a jet mill to reduce the particle size.

It is often desired to administer a drug, including a poorly water soluble drug, by subcutaneous or intravenous injection. If the drug is poorly soluble in water—typically the preferred vehicle for an injectable dosage form—the drug must be administered as a suspension or dispersion in which particle size is again an important consideration.

Thus, there is a need for a simpler and generally applicable means of making and delivering particles of drugs having a size below 10 μm and especially below 1 μm, especially for administration by inhalation or injection.

Cystic Fibrosis (CF) is a life shortening disorder that affects about 100,000 people worldwide. Much of the lung function loss is due to chronic infection of the lungs with pathogens such as Pseudomonas aeruginosa and others due to cycles of infection and inflammation. Constant treatment with antibiotics does not succeed in total eradication of the microorganisms and therefore leads to resistant strains. (L. Saiman et. al. Antimicrobial Agents and Chemotherapy, October 2001 p 2838-2844 and references therein). Delivering the drug orally usually can not lead to high enough drug concentrations in the target tissue. Direct pulmonary delivery of drugs by inhalation with agents such as tobramycin has given some improvement; however, neither the nebulizer formulations of tobramycin on the market, nor the experimental dry powder inhaler formulations are capable of reaching the deep lung with a sufficient amount of drug to effect a total eradication, thereby leading to resistance.

Cathelicidin peptides, are endogenous antimicrobial agents that have been shown to be effective at inhibiting CF pathogens. These peptides are being studied as agents for inhaled treatment of the lung infections. (Ibid). Peptide drugs are difficult to produce commercially, difficult to work with and their toxicity profile is unknown, especially for pulmonary delivery.

It has recently been shown (Tian-Tian Wang et. al. The Journal of Immunology 2004, 173; 2909-2912) that the administration of 1,25-dihydroxyvitamin D₃ (calcitriol) is an inducer of the antimicrobial peptide gene expression and as such could be a candidate for treating antibiotic-resistant pathogens such as Pseudomonas aeruginosa.

Calcitriol is well known for its effects on calcium homeostasis and is used to treat hypocalcaemia in doses of about 0.5 to 2 microgram. Larger doses of the drug can cause severe adverse effects of hypocalcaemia. On the other hand, for a sufficient dose to reach the lung and induce in-situ production of the antimicrobial peptides, oral delivery of the drug would need to be relatively high. There is therefore a need to bring calcitriol in sufficient concentration to the deep lung to induce antimicrobial peptides while minimizing systemic side effects.

While lung infections are usually treated through oral antibiotics, there has been considerable work in delivering such agents directly to the lungs through inhalation. One product that is available is a nebulizer formulation for tobramycin (PDR 60^{th} ed. 2006 page 1015). Work has also appeared in the literature for nebulizer formulations for Azithromycin (A. J. Hickey et al. Journal of Aerosol Medicine Volume 19 No. 1 2006 pg 54-60). Calcitriol is not particularly amenable to nebulizer formulations since it is very insoluble in water. One could conceivably formulate an emulsion and deliver it by nebulizer but then one needs the proper surface active agents which can be administered into the lung. Furthermore, calcitriol's dose is relatively low, making assurance of the stability and uniformity of the emulsion difficult. The low dose of calcitriol necessary for the induction of the antimicrobial peptide synthesis would make calcitriol a candidate for dry powder inhalation (DPI). Again two problems exist: Calcitriol's insolubility may make it unavailable once delivered and the need to deliver drug to the deep lung in sufficient quantities is always a problem with DPI.

Clearly, new methods for pulmonary dosing or administration of compounds like calcitriol that induce expression of genes encoding for antimicrobal peptides are needed.

### SUMMARY OF THE INVENTION

One aspect of the present invention relates to a pharmaceutical composition comprising a micronized pharmaceutical carrier bearing micronized drug microparticles.

Another aspect of the invention relates to a pharmaceutical composition for administration by inhalation comprising a pharmaceutical carrier bearing micronized drug microparticles, wherein the drug microparticles have a d₅₀ value of less than or equal to about 2 μm.

Another aspect of the invention relates to a pharmaceutical composition for administration by injection comprising a pharmaceutical carrier suitable for reconstitution into an injectable solution or suspension bearing non-mechanically micronized drug microparticles having a d₅₀ value of less than or equal to about 2 μm.

Another aspect of the invention relates to a method of making a pharmaceutical composition comprising the steps of: a) providing a solid solution of a drug and a sublimable carrier on the surface of a micronized pharmaceutical carrier particle, and b) subliming the sublimable carrier from the solid solution, thereby depositing micronized microparticles of the drug on the surface of the micronized pharmaceutical carrier particle.

Another aspect of the invention relates to a method of making a pharmaceutical composition comprising the steps of: a) forming a solid solution of a drug and a sublimable carrier on the surface of a micronized pharmaceutical carrier particle by applying a combination of the drug and molten sublimable carrier to the surface of at least one pharmaceutical carrier particle, and solidifying the combination by flash freezing to obtain the solid solution; and b) subliming the sublimable carrier from the solid solution to deposit micronized microparticles of the drug on the surface of the pharmaceutical carrier particle.

Another aspect of the invention relates to a pharmaceutical composition prepared by a process comprising the steps of: a) providing a solid solution of a drug and a sublimable carrier on the surface of a micronized pharmaceutical carrier particle, and b) subliming the sublimable carrier from the solid solution, thereby depositing micronized microparticles of the drug on the surface of the micronized pharmaceutical carrier particle.

In another aspect the invention relates to a pharmaceutical composition prepared by a process comprising the steps of: a) forming a solid solution of a drug and a sublimable carrier on the surface of a micronized pharmaceutical carrier particle by applying a combination of the drug and molten sublimable carrier to the surface of at least one pharmaceutical carrier particle, and solidifying the combination by flash freezing to obtain the solid solution; and b) subliming the sublimable carrier from the solid solution to deposit micronized microparticles of the drug on the surface of the pharmaceutical carrier particle.

Another aspect of the invention is a method of treating lung infection in cystic fibrosis by delivering a material that induces antimicrobial peptide gene expression to the lung by any of the methods of known inhalation therapy (pulmonary administration) including, for example, dry powder, metered dose, or nebulizer.

In another aspect of the invention, the inducer of peptide gene expression is present as microparticles with a diameter less than about 3000 nm.

In one aspect, the inducer is calcitriol.

Another aspect of this invention comprises a method of treating lung infection in cystic fibrosis by delivering an inducer to the lung in conjunction with an antibiotic agent or an antifungal agent by any of the methods of inhalation therapy.

In one aspect of the invention, the method comprises delivering calcitriol to the lung in conjunction with azithromycin.

In one aspect, the method comprises delivery by dry powder inhaler, wherein both the calcitriol and the azithromycin are present as particles with a diameter preferably less than 3000 nm, more preferably less than 1000 nm.

Another aspect of the invention comprises compositions of calcitriol for delivering calcitriol to the lung by dry powder inhaler, wherein the calcitriol is present as particles with a diameter preferably less than 3000 nm, more preferably less than 1000 nm.

Another aspect of this invention comprises a composition for pulmonary delivery including azithromycin, wherein the azithromycin is present as particles with a diameter preferably less than 3000 nm.

In one aspect, the calcitriol and/or antibiotic particles are not mechanically micronized. In one aspect, the particles are prepared by sublimation micronization.

Another aspect of the invention comprises a method for preparing azithromycin for pulmonary delivery comprising: (i) dissolving azithromycin in a sublimable solvent to form a solution; (ii) mixing the solution with a carrier; (iii) optionally adding at least one additional pharmaceutical additive; (iv) solidifying the solution to a solid solution on the carrier; and (v) subliming the sublimable solvent from the solid phase.

Another aspect of the invention comprises a composition including calcitriol wherein the calcitriol is present as particles with a diameter less than 3000 nm.

Another aspect of the invention comprises a composition including azithromycin wherein the azithromycin is present as particles with a diameter preferably less than 3000 nm.

Another aspect of this invention comprises a composition comprising azithromycin and calcitriol wherein the azithromycin and calcitriol are present as particles with a diameter less than 3000 nm.

### BRIEF DESCRIPTION OF THE FIGURES

FIG. 1 is a graph comparing the solubility of docetaxel that was prepared as a pharmaceutical composition according to the present invention to the solubility of a pharmaceutical composition containing docetaxel that was prepared by conventional means.

FIG. 2 is a bar graph showing the aerodynamic size distribution of beclomethason cyclocaps (400 μg) according to the present invention and as prepared by conventional means.

### DETAILED DESCRIPTION OF THE INVENTION

The present invention relates to a method of making a pharmaceutical composition using the technique of sublimation micronization. The general process of sublimation micronization is disclosed in copending and commonly owned U.S. patent application Ser. No. 10/400,100, the publication of which (US 2003/0224059) is incorporated herein in its entirety by reference. This publication includes the steps of forming a solid solution of a drug in a sublimable carrier, especially menthol, and removing the sublimable carrier from the solid solution by sublimation.

The present invention provides microparticles of a pharmacologically active substance, such as a drug, and a method for making drug microparticles. The invention also provides a drug delivery vehicle for administering a pharmacologically active substance, and methods for making such drug delivery vehicles, wherein the delivery vehicle includes at least one pharmaceutical carrier particle bearing microparticles of the drug.

The drug delivery vehicles of the invention are useful for oral delivery, inhalation delivery, nasal delivery, and injection delivery. Inhalation delivery includes dry powder inhalation, metered dose inhalation and nebulizer delivery.

Administration (delivery) by inhalation can be used for treatment of local lung conditions, that is where the situs of the disease is the lung, and it can be used as a method of delivering drugs to the entire system (systemic administration) through absorption in the lung. Compositions well suited for inhalation are those that exhibit desirable aerodynamic flow properties and possess drug particles having aerodynamic diameters that facilitate the entry and deposition in the desired portion of the lung.

Administration by injection (injection delivery) includes intravenous, subcutaneous, intramuscular, and intralesional injections. Compositions well suited for injection are those that are easily reconstituted into solution (such as in water, saline, or a water ethanol solution), and form a stable suspension.

Microparticles of the drug in the pharmaceutical of the present invention are formed as described hereinbelow and generally have mean dimensions on the order of about 50 nm up to about 10 μm. The drug microparticles preferably have a d₅₀ less than or equal to 3 μm, such as about 0.05, about 1, about 2, about 3 μm, and ranges made therefrom, such as about 0.05 to about 2, about 1 to about 3, etc. Microparticles according to the present invention can have a regular shape, e.g., essentially spherical, or they can have an irregular shape. The microparticles can be crystalline or can be at least partly amorphous. Preferably the microparticles are at least partly amorphous.

As used herein in connection with a measured quantity, the term “about” refers to the normal variation in that measured quantity that would be expected by the skilled artisan making the measurement and exercising a level of care commensurate with the objective of the measurement and the precision of the measuring equipment used.

Any pharmacologically active substance (drug) can be used in the practice of the present invention. However, drugs having poor water solubility (poorly water soluble drugs), and hence relatively lower bioavailability, are preferred and advantages of the present invention are more fully realized with poorly water-soluble drugs. For purposes of the present invention, a drug is considered to be poorly water soluble if it has a solubility of less than about 20 mg per milliliter of water. Examples of drugs having poor water solubility include fenofibrate, itraconazole, bromocriptine, carbamazepine, diazepam, paclitaxel, etoposide, camptothecin, danazole, progesterone, nitrofurantoin, estradiol, estrone, oxfendazole, proquazone, ketoprofen, nifedipine, verapamil, and glyburide, to mention just a few. Still further examples include docetaxel, other cytotoxic drugs, risperidone, beclomethasone, fluticasone, budesonide, other steroid drugs, salbutamol, terbutaline, ipratropium, oxitropium, formoterol, salmeterol, and tiotropium. The skilled artisan knows other drugs having poor water solubility. When administered by inhalation, preferred drug particles are non-toxic and are sufficiently soluble in the lung to provide efficacious levels of the drug in the plasma. When administered by injection, preferred carrier particles are non-toxic and totally soluble (i.e., at least 99% by weight) in the pertinent body fluid.

Pharmaceutical carrier particles useful for making the delivery vehicle of the present invention are made of comestible substances and are well known in the art. Preferred carrier particles are microparticulate. Examples of useful pharmaceutical carrier particles include particles, that can be non-pariel pellets, typically between about 0.1 mm and about 2 mm in diameter, and made of, for example, starch, particles of microcrystalline cellulose, lactose particles or, particularly, sugar particles. Suitable sugar particles (pellets, e.g. non-pariel 103, Nu-core, Nu-pariel) are commercially available in sizes from 35 to 40 mesh to 18 to 14 mesh.

For administration (delivery) by injection or inhalation routes according to preferred embodiments of the present invention, particles of lactose, dextran, dextrose, and mannitol are preferred pharmaceutical carriers for injection and inhalation uses, with lactose particles being most preferred. In a yet more preferred embodiment for inhalation administration, micronized lactose is used as the carrier for the drug particles which may be processed into the final product as is or further mixed with another pharmaceutical carrier before such processing. The skilled artisan knows other useful pharmaceutical carrier particles suitable for compositions to be administered by inhalation and/or injection.

In a particularly preferred embodiment, the micronized lactose has a particle size distribution, based on cumulative volume, of d₅₀ less than or equal to 10 μm, such as about 2 to 8, or about 6 to 7, and d₅₀ less than or equal to 15 μm, preferably less than or equal to about 10 μm. In another preferred embodiment the micronized lactose has a d₉₀ less than 5 μm. The terms “d₅₀” and “d₉₀” are well understood in the art. For example, a d₉₀ of 9 μm means that 90% (by volume) of the particles have a size less than or equal to 9 microns; a d₅₀ of 5 μm means that 50% (by volume) of the particles have a size less than or equal to 5 microns, as tested by any conventionally accepted method such as the laser diffraction method. d₅₀ and d₉₀ values can be determined by various techniques known in the art, such as laser diffraction. Suitable methods for laser diffraction, for example, are well known and can be obtained from various sources, such as from Malvern Instruments (U.K.). As used herein, the phrase “average particle size” refers to the d₅₀ value.

In the Examples provided herein, d₅₀ and d₉₀ values for lactose were obtained using a Malvern Mastersizer 2000 equipped with a Hydro 2000S measuring cell, with the appropriate refractive index for lactose (i.e., 1.5) in ethanol solvent (refractive index 1.36). One of ordinary skill in the art would understand that the particular parameters used in measuring particle size by laser diffraction, such as the particle refractive index, dispersant refractive index, and absorption value depend on the solvent being used and the specific particle being measured. For example, when measuring the particle size of a fluticasone and lactose formulaton via laser diffraction, using water as a solvent, the particle refractive index is 1.500, absorption is 0, and the dispersant refractive index is 1.330. Lactose particles with suitable d₅₀ and d₉₀ values are commercially available as, e.g., Lactohale®, from Friesland Food Domo.

The attaching of the sub-micron particles to the micronized lactose prevents the drug particles from being exhaled during respiration, while making the drug more readily available for local action and systemic absorption due to enhanced dissolution properties. For most applications, the optimal size of the sub-micron particles attached to the micronized carrier provides enough kinetic energy to prevent exhalation of the drug particles during respiration, yet not so much kinetic energy that the particles deposit in the major airways (i.e., the bronchi) rather than the lung.

The microparticles of the drug or pharmacologically active substance of the present invention are preferably obtained by removing a sublimable carrier from a solid solution of the drug in the sublimable carrier. The drug or pharmaceutically active substance can be present with the sublimable carrier in the solid solution as discrete molecules, or it can be present in aggregates of a few hundred, a few thousand, or more molecules. The drug need only be dispersed on a sufficiently small scale so that sufficiently small, discrete microparticles are ultimately obtained. Preferably, the drug or pharmacologically active substance in the solid solution is dissolved in the sublimable carrier.

Preferred sublimable carriers useful in the practice of the present invention form solid solutions with the drug at an easily accessible temperature and can be removed from the solid solution without heating the solid solution to a temperature above the melting point of the solid solution, for example by sublimation. Sublimable carriers have a measurable vapor pressure below their melting point. Preferred sublimable carriers have a vapor pressure of at least about 10 Pascal, more preferably at least about 50 Pascal at about 10° or more below their normal melting points. Preferably, the sublimable carrier has a melting point between about −10° C. and about 200° C., more preferably between about 20° C. and about 60° C., most preferably between about 40° C. and about 50° C. Preferably, the sublimable carrier is a substance that is classified by the United States Food and Drug Administration as generally recognized as safe (i.e., GRAS). Examples of suitable sublimable carriers include menthol, thymol, camphor, t-butanol, trichloro-t-butanol, imidazole, coumarin, acetic acid (glacial), dimethylsulfone, urea, vanillin, camphene, salicylamide, and 2-aminopyridine. Menthol is a particularly preferred sublimable carrier.

The solid solutions of the present invention can exist as a true homogeneous crystalline phase of the interstitial or substitutional type, composed of distinct chemical species occupying the lattice points at random, or they can be a dispersion of discrete molecules or aggregates of molecules in the sublimable carrier.

The solid solutions can be made by combining a drug with molten sublimable carrier, then cooling the combination to below the melting point of the solid solution.

Preferably, the solid solution is formed by combining the drug with molten sublimable carrier, applying the combination to at least one pharmaceutical carrier particle, preferably a micronized pharmaceutical carrier particle, and allowing the combination to solidify to obtain the solid solution on the surface of the pharmaceutical carrier particle.

Solidification is preferably accomplished by flash freezing. Flash freezing preferably includes mixing liquid nitrogen with the combination of drug and molten sublimable carrier that is on the surface of the pharmaceutical carrier particle. Alternatively, flash freezing preferably includes pouring the combination of drug and molten sublimable carrier that is on the surface of the pharmaceutical carrier particle into liquid nitrogen. In a most preferred embodiment, a stream of the pharmaceutical carrier particles bearing the combination of drug and sublimabal carrier is concurrently flowed with a stream of liquid nitrogen onto the screen of a pharmaceutical mill. The combination of drug and sublimable carrier that is deposited on the pharmaceutical carrier particles is flash frozen, and the product is milled immediately thereafter.

The solid solutions can also be formed by combining a drug and a sublimable carrier in an organic solvent and evaporating the organic solvent to obtain a solid solution of drug in sublimable carrier. Ethanol is an example of a preferred organic solvent that can be used in the practice of the present invention.

The solid solution can also include a compound or polymer that forms a dispersion with the drug. Preferred compounds that may be added to the solid solution include, surface active agents, hydroxypropylcellulose, polyethylene glycols (PEG), and poloxamer of such grade and amount that allow the sublimable carrier to solidify at reasonable temperatures. In a preferred embodiment, PEG 1000 or above is used with or without added poloxamer. In a more preferred embodiment, PEG 6000 or poloxamer 407 is used, and in a most preferred embodiment, both PEG 6000 and poloxamer 407 are used in the formulation.

In a preferred embodiment, the solid solution is formed on the surface of at least one pharmaceutical carrier particle and preferably a plurality of pharmaceutical carrier particles, still more preferably on a plurality of micronized pharmaceutical carrier particles. For example, a molten combination of drug and carrier can be applied to the surface of a pharmaceutical carrier particle where it is allowed to cool to form the solid solution on the surface of the pharmaceutical carrier particle. A solid solution can also be formed at the surface of a pharmaceutical carrier particle by applying a combination of solvent, drug, and sublimable carrier to at least one, and preferably a plurality of, pharmaceutical carrier particle(s) and evaporating the organic solvent to obtain the solid

When no solvent is used, application is at a temperature above the melting point of the sublimable carrier. When drug and sublimable carrier are combined with solvent, application is at a temperature such that drug and sublimable carrier remain in solution in the solvent.

The microparticles of the present invention are formed by removal of sublimable carrier from a solid solution, made as described above, at a temperature below the melting point of the solid solution. The solid solution should be kept at a temperature below its melting point to preserve the solid solution during the process of removing the sublimable carrier. The sublimable carrier can be removed from the solid solution by, for example, treating the solid solution, deposited on a pharmaceutical carrier particle where applicable, in a stream of air, preferably heated air, in, for example, a fluidized bed drier.

Removal of sublimable carrier from the solid solution, whether coated on a pharmaceutical carrier particle or not, results in formation of the microparticles of the present invention.

In another embodiment of the present invention, the microparticles of drug or the pharmaceutical carrier particles bearing microparticles of a drug are formulated into pharmaceutical compositions that can be made into dosage forms, in particular oral solid dosage forms such as capsules and compressed tablets, as are well known in the art, capsules or other receptacles for inhalable dosage forms in dry powder inhalers, metered dose inhalers, or nebulizers, powders, powder beds or granules in vials or other receptacles for reconstitution into injectable solutions or suspensions, and reconstituted solutions or suspensions for injections. The injections may be for intravenous, subcutaneous, intramuscular or intralesional injections.

Pharmaceutical carrier particles bearing microparticles of a drug made in accordance with the present invention have excellent bulk flow properties and can be used directly, alone or in combination with carrier particles that do not carry a drug, to make capsule dosage forms. If necessary, diluents such as lactose, mannitol, calcium carbonate, and magnesium carbonate, to mention just a few, can be formulated with the microparticle-bearing pharmaceutical carrier particles when making capsules.

In describing inhalation formulations, it is often useful to refer to the “aerodynamic diameter” of a particle. As used herein, the aerodynamic diameter refers to the behavioral size of the particles of an aerosol. Specifically, it is the diameter of a sphere of unit density which behaves aerodynamically like the particles of a test substance. The aerodynamic diameter is used to compare particles of different sizes, shapes, and densities and to predict where in the respiratory tract such particles may be deposited. This term is used in contrast to “optical,” “measured” or “geometric” diameters which are representations of actual diameters which in themselves do not determine deposition within the respiratory tract.

In describing the aerodynamic size distribution and/or particle size distribution of a formulation, the mass median aerodynamic diameter (“MMAD”) represents the number wherein fifty percent of the particles by weight will be smaller than the mass median aerodynamic diameter and 50% of the particles will be larger. The geometric standard deviation (“GSD”) refers to a dimensionless number equal to the ratio between the MMAD and either 84% or 16% of the diameter size distribution (e.g., MMAD=2 m; 84%=4 m; GSD=4/2=2.0). The MMAD, together with the GSD, can be used to describe the particle size distribution of an aerosol statistically, based on the weight and size of the particles. Suitable methods and devices for measuring aerodynamic size distribution are well known in the art, such as by multi-stage liquid impinger (MSLI).

In the Examples provided herein, the aerodynamic size distributions were obtained using a MSP Corp. New Generator Impactor (NGI), supplied by Copley Scientific, set at a flow of 100 liters/min. with a sampling duration of 2.4 seconds, together with a PCH Cyclohaler.

The fine particle dose (“FPD”) refers to the amount of an active pharmaceutical ingredient present in the fine particles (generally, less than 5 μm) in a delivered dose as indicated, for example, in a MSLI or NGI test.

The fine particle fraction refers to the ratio of the fine particle dose to the delivered dose. It is this fraction (or percent) of an active pharmaceutical ingredient in a dose that is generally presumed by those of ordinary skill in the art to reach the deep lung.

The present invention further provides a combination for pulmonary delivery for treating, by inhalation therapy, an opportunistic lung infection in a cystic fibrosis patient suffering from such lung infection, which combination includes microparticles, especially microparticles having mean dimensions of about 3000 nm, preferably less than about 1000 nm, of a vitamin D compound, especially calcitriol or a prodrug thereof deposited or carried on pharmaceutical carrier particles. The combination preferably also includes an antifungal agent or antimicrobal agent.

The invention also provides combinations of microparticles of compounds, referred to herein as inducer compounds, capable of inducing the in vivo expression of genes, preferably human genes, that encode for antimicrobal peptides; pharmaceutical carrier particles; and, optionally at least one of an antimicrobal agent or an antifungal agent, or both. The combination can be used as such or as part of a pharmaceutical composition that it is capable of delivering to the lung the inducer compound in the form of microparticles, preferably smaller than 3000 nm and more preferably smaller than 1000 nm, larger particles being decreasingly less effective.

The combinations can also contain other components, such as additives to stabilize the combination or any part thereof during manufacturing or storage, antioxidants being an example. The combinations can also include or be formulated into pharmaceutical compositions with pharmaceutically acceptable excipients.

The skilled artisan knows of many compounds capable of inducing expression of genes that encode for antimicrobal proteins, all of which are within the scope of the present invention. Vitamin D compounds, especially calcitriol or analogs or prodrugs thereof that are capable of inducing expression of genes encoding for antimicrobal proteins are preferred inducer compounds in the practice of the present invention.

Calcitriol has the following structure:

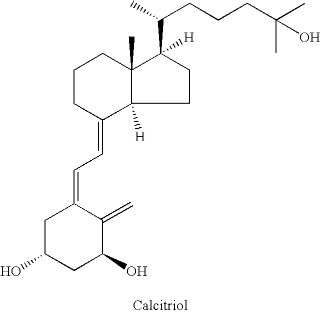

In some embodiments, the inducer compound, preferably calcitrol, is present in the combination as microparticles, preferably smaller than 3000 nm and more preferably smaller than 1000 nm in size, preferably formed by sublimation micronization.

Since calcitriol induces gene expression for forming antimicrobial peptides there may be a delay in onset of action of antibiotic activity. There may also be opportunistic fungal infections underlying the microbial infection. Therefore, in certain embodiments of the invention one combines the calcitriol for delivery to the lung with an antibiotic or an antifungal agent. In certain embodiments, the combination includes an antimicrobal agent like those known in the art. Azithromycin is a preferred antimicrobal agent for use in this and other embodiments of the invention.

The method of treating a lung infection in cystic fibrosis includes delivering calcitriol to the lung by any of the methods of inhalation, e.g., dry powder, metered dose, or nebulizer. In a preferred embodiment of this invention, calcitriol would be delivered as nanoparticles, i.e., particles smaller than 3000 nm or more preferably particles smaller than 1000 nm. The smaller particles are expected to carry deeper into the lung and treat parts of the lung not accessible to nebulizer treatment. At the same time, the smaller particles will allow the calcitriol to dissolve within the lung whereas larger particles will be less soluble or mostly insoluble. However, producing calcitriol having the particle sizes described is not a simple task considering the sensitivity of calcitriol to degradation by the environment and handling.

The combinations of the present invention can be made by the process of sublimation micronization, described above. This method is particularly advantageous for use with inducers like calcitrol that are easily degraded by light, oxygen, and especially heat.

Sublimable solvents and pharmaceutical carrier particles suitable for use in the method of the invention are described above. Lactose is a preferred carrier particle in this embodiment of the invention, and may have a particle size in the range of 5 μm to 500 μm, more preferably about 50 to 150 μm.

In a preferred embodiment, the combination includes both an inducer compound, e.g., calcitriol, and an antimicrobal compound, e.g., azithromycin. In a more preferred embodiment the calcitriol and azithromycin are prepared for DPI by dissolving the two drugs together in a sublimable solvent and carrying out sublimation micronization on lactose or other acceptable excipient carrier, so that both drugs are present as nano scale drugs. In a more preferred embodiment, both drugs are present in a size of less than 3000 nm, more preferred less than 2000 nm and most preferred less than about 1000 nm. In one preferred embodiment, antioxidants are added to the formulation and in another preferred embodiment, acceptable surface active agents are added alone or with the antioxidants.

In another embodiment, the present invention provides a combination or composition of calcitriol for delivering calcitriol to the lung by dry powder inhaler. In one embodiment the calcitriol is deposited on an acceptable carrier material such as lactose. The pharmaceutical carrier may be micronized, or may be in a mixture with micronized carrier. The dose of calcitriol is preferably 0.1 to 10 microgram, more preferably 0.5 to 5 microgram and most preferably about 2 micrograms of calcitriol. In a preferred embodiment, the calcitriol is present as particles with a diameter of less than 3000 nm and in a more preferable embodiment the particle size is less than 2000 nm and most preferably less than 1000 nm. A preferable method of preparing the calcitriol on the pharmaceutical carrier is by sublimation micronization as mentioned above. In a preferred embodiment the composition further comprises an antibiotic or an antifungal agent. In a more preferred embodiment the antibiotic is also in particles of less than 3000 nm, less than 2000 nm or less than 1000 nm. In a more preferred embodiment the antibiotic agent is azithromycin. In a most preferred embodiment the calcitriol and the azithromycin are sublimation micronized together on lactose wherein both have an average particle size of less than 1000 nm. The preferred dose of calcitriol is 0.1 to 10 microgram, more preferably 0.5 to 5 microgram and most preferably about 2 micrograms of calcitriol while the preferred dose of azithromycin is 5 to 20 mg and most preferable about 10 to 15 mg. Antioxidants and surface active agents are optional additives.

The combinations of the invention can also include other additives. These optional pharmaceutical additives include antioxidants and surface active agents, i.e., compounds that modify properties like surface tension and contact angle in a manner improving the suitability of the combination or pharmaceutical composition containing it for inhalation administration. In a preferred embodiment of the invention, the solidification step is preferably accomplished by flash freezing the solution by mixing with liquid nitrogen or pouring into liquid nitrogen. In a most preferred embodiment of the invention, a stream of the molten mix of carrier with molten solvent in which the calcitriol and other additives are dissolved is concurrently flowed with a stream of liquid nitrogen onto the screen of a pharmaceutical mill. The molten solvent is flash frozen and the product milled immediately thereafter. In a most preferred embodiment, an antibiotic or anti fungal agent is added to the molten sublimable solvent along with the calcitriol. In a most preferred embodiment this antibiotic is azithromycin.

In another embodiment, the invention comprises a composition including azithromycin wherein the azithromycin is present as particles with a diameter preferably less than 3000 nm. The present invention also comprises a combination or composition of azithromycin for delivering azithromycin to the lung by dry powder inhaler. In one embodiment the azithromycin is deposited on an acceptable carrier material, such as lactose. The pharmaceutical carrier may be micronized, or may be in a mixture with micronized carrier.

The following numbered embodiments exemplify some of the preferred embodiments of the invention:

In a First embodiment, the invention relates to a combination for pulmonary delivery for treating, by inhalation therapy, an opportunistic lung infection in a cystic fibrosis patient suffering from such lung infection which combination includes microparticles, especially microparticles having mean dimensions of about 3000 nm, preferably less that about 1000 nm, of a vitamin D compound, especially calcitriol or a prodrug thereof deposited or carried on pharmaceutical carrier particles. The combination can and preferably does also include an antifungal agent or antimicrobal agent.

In a Second embodiment, the present invention provides a combination according to the First embodiment wherein the vitamin D compound is calcitriol, also known as 1,25-dihydroxycholecalciferol.

In a Third embodiment, the present invention relates to a combination of either of the first or second embodiments in which the microparticles are formed by the process of sublimation micronization whereby the microparticles are formed by subliming the sublimable carrier, especially menthol, t-butanol, or a mixture of menthol and t-butanol, from a solid solution of the vitamin D compound and, optionally, one or more antimicrobal agent, antibacterial agent, antifungal agent or combination thereof, in the sublimable carrier.

In Fourth and Fifth embodiments, the present invention relates to a combination of the Third embodiment in which the sublimable carrier is menthol and includes an antimicrobal agent, especially azithromycin (Fourth embodiment) or includes an antifungal agent (Fifth embodiment).

In a Sixth embodiment, the present invention provides a combination according to any of the First through Fifth embodiments in which the carrier particles are sugar particles, preferably lactose particles.

In a Seventh embodiment, the present invention relates to a method of treating an opportunistic lung infection in a patient having cystic fibrosis and suffering from such opportunistic lung infection by administering to the patient a combination of any embodiment of the invention, either alone or in a pharmaceutical composition.

In an Eighth embodiment, the present invention provides a method of making a combination suitable for administration by inhalation to a mammal, especially a human suffering from cystic fibrosis, the combination being effective for treating opportunistic lung infection, the method including the steps of providing a solid solution of a vitamin D compound, preferably calcitriol, in a sublimable carrier, preferably menthol, which solid solution optionally contains an antimicrobal agent, an antifungal agent, or both; and removing the sublimable carrier by sublimation.

In a Ninth embodiment, the present invention provides a method of the Eighth embodiment in which the solid solution provided is obtained by flash-freezing, for example by combining molten solution with liquid nitrogen or solid carbon dioxide, which itself sublimes. Other compounds that induce expression of genes encoding for antimicrobal peptides can be used in place of the vitamin D compound in the present invention in any of its embodiments.

The present invention is further illustrated with the following non-limiting examples.

### EXAMPLE 1

### Solubility of Selected Drugs in Menthol

The following general procedure was repeated with several drugs with menthol carrier.

Menthol (10 grams) was melted on a stirring hot plate with magnetic stirring, then heated to the desired temperature indicated in Table 1. The desired drug was added in small increments (approximately 0.1 grams) and stirred to obtain a clear solution. The desired drug was added in increments until no more drug dissolved in the menthol. The weight of material added to the menthol melt that still gave a clear solution was taken as the solubility of the active drug at the indicated temperature. The results are given in Table 1.

TABLE 1
Solubility of selected active drug substances in menthol

| | | Solubility |
|---|---|---|
| Active drug substance | temperature (° C.) | (% w/w) |
| Azithromycin | 63 | 40.0 |
| Cyclosporin | 55 | 39.2 |
| Diazepam | 43 | 5.7 |
| Fenofibrate | 60 | 37.5 |
| Itraconazole | 61 | 1.0 |
| Oxybutynin | 60 | 9.1 |
| Risperidone | 70 | 8.3 |
| Salicylic acid | 43 | 16.0 |
| Simvastatin | 63 | 30.0 |

### EXAMPLE 2

### Improvement of the Dissolution of Fenofibrate by “Menthol Micronization”

Menthol (50 grams) was heated in a jacketed reactor to 60° C. After melting, the melt was stirred at 100 rpm. Fenofibrate (25 grams) was added and the mixture stirred at 100 rpm and 60° C. until full dissolution was achieved. Microcrystalline cellulose (Avicel ph 102, 55 grams) was added to the melt and the mixture was stirred for 30 minutes. The heat source was then removed and the mass allowed to cool to room temperature with the stirring continued at 100 rpm for a further 30 minutes.

The obtained mass was milled through a 6.35 mm screen in a Quadro Comil mill at 1300 rpm. The milled product was allowed to cool to 25° C. and milled again through 1.4 mm screen to obtain a powder in which the fenofibrate is dissolved in menthol and coated on the microcrystalline cellulose.

The powder was transferred to a fluid bed dryer (Aeromatic model STREA1) where the menthol was removed by drying for three hours at 30-32° C. with the fan at 7-8 Nm³/hr. A powder, 62 grams, was obtained. This powder was a micronized fenofibrate deposited on microcrystalline cellulose.

A sample of this powder containing 100 mg of the fenofibrate was tested for dissolution in a USP Apparatus II dissolution tester in 900 ml 0.5% sodium lauryl sulfate (SLS) in water at 37° C. and 100 rpm. The fenofibrate in the dissolution medium was determined by HPLC on an Hypersil® ODS column with UV detection at 286 nm. The results are shown in Table 2. Fenofibrate micronized by the menthol method gave 100% dissolution in two hours. An equivalent simple combination of fenofibrate (control, not deposited from menthol) with microcrystalline cellulose gave 40.2% dissolution in 3 hours, while a mechanically micronized fenofibrate raw material mixed with microcrystalline cellulose gave 72.1% dissolution in 3 hours.

TABLE 2
Dissolution of menthol treated fenofibrate

| time (minutes) | % dissolved |
|---|---|
| 15 | 44.0 +/− 1.3 |
| 30 | 73.6 +/− 2.9 |
| 60 | 82.3 +/− 0.6 |
| 90 | 93.1 +/− 4.2 |
| 120 | 102.7 +/− 0.2 |
| 180 | 104.9 +/− 0.8 |

### EXAMPLE 3

### Improvement of the Dissolution of Oxybutynin Chloride by “Menthol Micronization”

Menthol (80 grams) was melted and oxybutynin chloride (8 grams) and microcrystalline cellulose (89.5 grams) were added and treated as in Example 2 to give a powder of micronized oxybutynin chloride on microcrystalline cellulose.

The dissolution of oxybutynin chloride from this powder (a sample of powder containing 100 mg of the active drug) was tested in a USP apparatus II dissolution tester in 100 ml of 50 mM phosphate buffer pH=6.8 at 37° C. and 50 rpm. The oxybutynin content of the dissolution sample was measured by spectrophotometer at 225 nm. The results are given in Table 3. The dissolution reached 79.2% at three hours. An equivalent simple combination of the oxybutynin chloride raw material with microcrystalline cellulose that was not treated with the menthol micronization method gave only 22.1% dissolution in three hours.

TABLE 3
Dissolution of menthol treated oxybutynin

| time (minutes) | % dissolved |
|---|---|
| 30 | 21.5 +/− 0.4 |
| 90 | 59.7 +/− 1.2 |
| 180 | 79.2 +/− 1.0 |

### EXAMPLE 4

### Improvement of the Dissolution of Risperidone by Menthol Micronization

Menthol (50 grams) was melted and risperidone (4.5 grams) and microcrystalline cellulose (62.5 grams) were added and treated according to the procedure in Example 2. A sample of the resulting powder (containing 50 mg of risperidone ) was tested in a USP apparatus II dissolution tester using 900 ml of water at 37° C. and 100 rpm. The concentration of risperidone in the dissolution samples was measured using a spectrophotometer at 240 nm.

The results of the dissolution of the menthol micronized powder and of the control simple combination of risperidone and microcrystalline cellulose (not treated with menthol) are shown in Table 4. The menthol deposited risperidone gave 100% dissolution in 30 minutes, whereas the control mixture gave 31.9% in thirty minutes and 63.7% in three hours.

TABLE 4
Dissolution of menthol treated risperidone vs. control

| time (minutes) | % dissolved test | % dissolved control |
|---|---|---|
| 15 | 69.3 +/− 0.5 | 17.5 +/− 2.6 |
| 30 | 99.9 +/− 1.0 | 31.9 +/− 3.5 |
| 60 | 102.3 +/− 0.8 | 41.7 +/− 5.6 |
| 90 | 102.8 +/− 1.2 | 48.2 +/− 8.3 |
| 120 | | 53.2 +/− 11.1 |
| 180 | | 63.7 +/− 8.3 |

### EXAMPLE 5

### Improvement of the Dissolution of Cyclosporin by Menthol Micronization

Menthol (80 grams) was melted and cyclosporin (20 grams) and microcrystalline cellulose (100 grams) were added and treated as in Example 2. A sample of this powder (containing 10 mg of menthol-micronized cyclosporin) was tested for dissolution in 900 ml water in a USP apparatus II dissolution unit at 37° C. and 100 rpm. The cyclosporin content of the dissolution samples was determined spectrophotometrically at 215 nm. The dissolution of the menthol deposited material and of a control mixture of cyclosporin and microcrystalline cellulose (not deposited from menthol) are presented in Table 5.

The cyclosporin dissolution from the powder having cyclosporin deposited from menthol was about twice that of the control (simple combination), and the maximum dissolution was achieved in shorter time.

TABLE 5
Dissolution of menthol treated cyclosporin vs. control

| time (minutes) | % dissolved test | % dissolved control |
|---|---|---|
| 30 | 9.2 +/− 0.3 | 0.1 +/− 0.0 |
| 60 | 11.9 +/− 0.3 | 1.3 +/− 0.5 |
| 90 | 13.1 +/− 0.5 | 3.1 +/− 0.2 |
| 120 | 13.3 +/− 0.3 | 5.1 +/− 0.2 |
| 180 | 14.3 +/− 0.8 | 7.1 +/− 0.3 |

### EXAMPLE 6 (COMPARATIVE)

### Attempted Improvement in Itraconazole Dissolution by Menthol Micronization

Menthol (92 grams) was melted as in Example 2. Itraconazole (3.6 grams) was added and mixed well in the melt. A solution was not formed because itraconazole has a solubility of only 1% in menthol at 60° C. (see Table 1). To the suspension of itraconazole in menthol was added microcrystalline cellulose (90 grams) and the mixture treated as in Example 2. The dissolution of the itraconazole was measured from a powder sample containing 100 mg of the drug in 900 ml of 0.1N HCl in a USP apparatus II dissolution tester at 37° C. and 100 rpm. The dissolved itraconazole was measured spectrophotometrically at 251 nm. The results of the dissolution are shown in Table 6. The dissolution was about 8% at 30 minutes and the same at three hours. A control simple mixture of itraconazole and microcrystalline cellulose (not deposited from menthol) gave essentially the same results (7.8% in three hours).

TABLE 6
Dissolution of menthol treated itraconazole

| time (minutes) | % dissolved |
|---|---|
| 30 | 8.8 +/− 0.4 |
| 90 | 8.0 +/− 0.6 |
| 180 | 8.1 +/− 0.1 |

### EXAMPLE 7

### Dissolution of Menthol-Micronized Docetaxel

Menthol (5.0 gm) was melted on a hot plate. PEG 6000 (50 mg) and Poloxamer 407 (50 mg) were added and a homogenous solution obtained. Docetaxel (100 mg) was added and fully dissolved in the mixture. (n.b. Docetaxel is soluble in the menthol melt without the additives so one may, if so desired, change the order of addition and first dissolve the docetaxel in the menthol and subsequently add the PEG6000 and Poloxamer 407.) Lactose (1.0 gm) was added and stirred to obtain an approximately homogenous suspension. The so obtained suspension was placed in a freezer to obtain a solid solution mixed with the lactose carrier. Another sample was prepared where microcrystalline cellulose was used in place of the lactose. After coarse mechanical milling the solid was placed in a vacuum oven or a lyophilizer and the menthol removed at temperatures between 20 and 40 degrees. A powder was obtained of the menthol-micronized docetaxel on the lactose or microcrystalline cellulose.

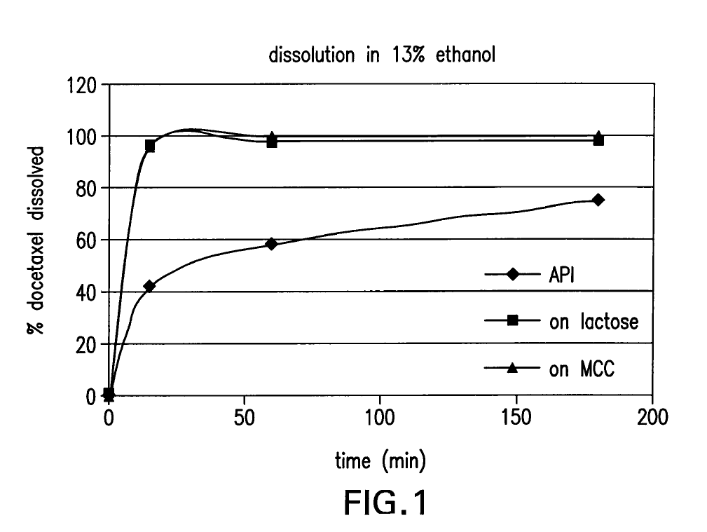

The dissolution of docetaxel from these powders was tested against the dissolution of the docetaxel API granulated with 2% PVP on lactose. The dissolution was measured in 900 ml 13% ethanol in water in a USP apparatus II dissolution tester at 37° C. and 50 rpm. The results are given in Table 7 and FIG. 1.

TABLE 7
% Docetaxel Dissolved in 13% ethanol in water

| time | | on | |
|---|---|---|---|
| (min) | API | lactose | on MCC |
| 0 | 0 | 0 | 0 |
| 15 | 42 | 96 | 96 |
| 60 | 58 | 98 | 100 |
| 180 | 75 | 98 | 100 |

### EXAMPLE 8

### Inhalable Formulation of Beclomethasone Made Using Menthol Micronization

In the experiment described here, menthol micronization is performed for the manufacturing of beclomethasone cyclocaps 400 μg. In the regular production process, the micronized active ingredient is mixed in a high shear mixer with lactose monohydrate, which is used as a carrier. The powder mixture is filled in hard shell capsules.

The aerodynamic assessment of fine particles of the product manufactured in accordance with the regular process will be compared with capsules containing beclomethasone raw material obtained after menthol micronization. The following materials were used in the experiment.
Beclomethasone dipropionate, Sicor Italy, batch P304736, laser particle) size distribution: d₁₀=1 μm, d₅₀=2 μm, d₉₀=3 μm;
Lactose monohydrate Microfine, Borculo The Netherlands, laser particle size distribution: d₅₀=5 μm, d₉₀=9 μm.
Lactose monohydrate DMV The Netherlands, broad distribution.

The general procedure that is employed follows. The specific working example is given thereafter.

General Procedure:

Melt L-menthol using a water bath at 50° C. Dissolve the beclomethasone raw material in the melted menthol. Add micronized lactose monohydrate (Microfine, Borculo) and mix until homogeneous. Cool the suspension to room temperature. Mill the obtained mixture. Remove the menthol from the mixture by sublimation in the lyophilizer.

Prepare one batch of Beclomethasone cyclocaps 400 μg with the micronized lactose monohydrate bearing beclomethasone particles obtained after menthol micronization. Complete the formulation with the regular cyclolactose mixture (lactose monohydrate DMV). Total batch size 400 g (=16,000 capsules).

Fill the powder mixture into size 3 hard shell capsule. Seal the capsules. Determine the assay and the fine particle dose (FPD) of both formulations. Compare the results.

A recapitulation of the particular experimental detail follows.

### SPECIFIC WORKING EXAMPLE

75.0 g L-Menthol was melted at 50° C. using a water bath. An amount of 7.5 g beclomethasone dipropionate was weighed and dissolved in the melted menthol. After a clear solution was obtained, 40.8 g of micronized lactose monohydrate was dispersed. The suspension was allowed to solidify at room temperature and was subsequently milled using a grated screen (1.5 mm). The powder was filled into glass trays and placed in the lyophilizer. The menthol was sublimed using the program as described in Table 8.

TABLE 8
Lyophilisation program for menthol sublimation

| | Temperature | Vacuum | Time | |
|---|---|---|---|---|
| | (° C.) | (mTorr) | (min) | Ramp/Hold |
| Load | 20 | — | — | — |
| Step #1 | 30 | 150 | 30 | H |
| Step #2 | 35 | 150 | 60 | R |
| Step #3 | 35 | 150 | 720 | H |
| Step #4 | 40 | 150 | 60 | R |
| Step #5 | 40 | 150 | 960 | H |
| Post Heat | 40 | 50 | 30 | — |

Preparation of batch ID 601.16: The lyophilized beclomethasone/micronized lactose monohydrate mixture was mixed in a high shear mixer with the regular cyclolactose (non-micronized) mixture. All components were previously sieved through a 0.7 mm screen before mixing. The powder mixture was filled in size 3 gelatin capsules. Each capsule contained 25 mg of powder mixture. The composition of the product is stated in Table 9. The capsules were sealed with a gelatin band and stored for 24 hours at 25° C./60% RH

Preparation of batch ID 601.015, Beclomethasone cyclocaps 400 μg: A regular beclomethasone mixture was made with additional micronized lactose monohydrate to compensate for the amount of micronized lactose used in the menthol micronization process. The active ingredient was first manually mixed with the micronized lactose monohydrate followed by high shear mixing with the regular cyclolactose. All components were sieved through a 0.7 mm screen prior to mixing. The size 3 gelatin capsules were filled with 25 mg of powder mixture. After sealing the capsules were stored for 24 hours at 25° C./60% RH.

TABLE 9
Composition per capsule of Beclomethasone cyclocaps 400 μg

| | | Beclomethasone |
|---|---|---|
| | Beclomethasone | Cyclocaps 400 μg |
| | Cyclocaps 400 μg | 601.016 |
| | 601.015 | ‘Menthol |
| Component | ‘Regular’ | micronized’ |
| Beclomethasone menthol | — | 2.96 mg |
| micronized/ lactose | | |
| monohydrate, micronized* | | |
| Beclomethasone dipropionate (not | 0.460 mg | — |
| menthol micronized) | | |
| Lactose monohydrate, micronized | 2.50 mg | — |
| Lactose monohydrate | 22.07 mg | 22.07 mg |
| Total weight | 25.0 mg | 25.0 mg |

*Contains 0.460 mg beclomethasone dipropionate and 2.50 mg micronized lactose monohydrate

The assay and fine particle dose (FPD) of both batches were determined.

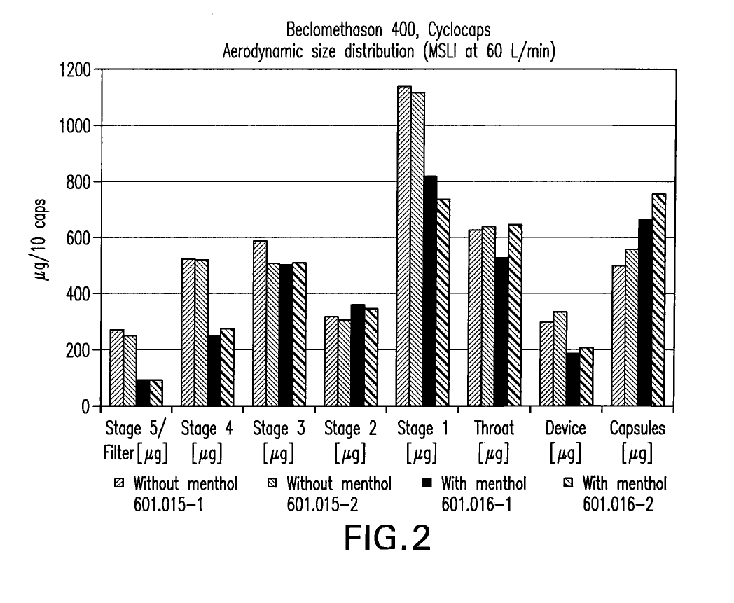

FIG. 2 shows the aerodynamic size distribution in duplicate of both batches. Table 10 gives analytical results for both batches. The aerodynamic size distributions were obtained using a MSP Corp. New Generator Impactor (NGI), supplied by Copley Scientific, set at a flow of 100 liters/min. with a sampling duration of 2.4 seconds, and a PCH Cyclohaler.

The assay of the capsules containing the menthol micronized active is somewhat low. This may be due to inexperience with the preparation of the menthol solution. For this reason the fine particle dose of these capsules is also lower. However, the assay demonstrates the feasibility of the method.

The results show that the FPD is also limited by the particle size distribution (PSD) of the micronized lactose. The beclomethasone raw material may be strongly attached to the lactose.

TABLE 10
Analytical results of Beclomethasone cyclocaps 400 μg
batch 601.015 and 601.016

| | Beclomethason | Beclomethason |
|---|---|---|
| | Cyclocaps 400 μg | Cyclocaps 400 μg |
| | 601.015 | 601.016 |
| Parameter | ‘Regular’ | ‘Menthol’ |
| Average mass fill weight (mg) | 24.0 | 25.1 |
| Assay¹ (%) | 107.4 | 90.4 |
| Fine particle dose (%) | 33.2 | 21.5 |
| MMAD² (μm) | 3.3 | 4.6 |
| GSD³ | 2.2 | 2.0 |
| Delivered dose, | 85.1 | 64.4 |
| based on label claim (μg) | | |
| Fine particle fraction, | 39.0 | 33.4 |
| based on calculated | | |
| delivered dose (%) | | |

¹An overage of 15% is used.
²MMAD refers to mass median aerodynamic diameter.
³“GSD” refers to geometric standard deviation.

### EXAMPLE 9

### Comparative Lung and Systemic Delivery of Fluticasone delivered by Dry Powder Inhaler (DPI) in Beagle Dogs

A: Production of Fluticasone Propionate on Lactose

To 100 g melted menthol (60° C.), 0.5 g HPC LF was added. The mixture was stirred until a clear solution was formed. To this heated solution, 0.5 g Fluticasone propionate (Teva API-Sicor Mexico) powder was added and the solution was stirred for 2 hours until an almost clear solution was formed. 4.0 g of micronized lactose powder (Teva API d(0.1) 1.99μ, d(0.5) 6.65μ, d(0.9) 14.63μ) was added in and stirred for 10 minutes until a homogenous suspension of the lactose was obtained.

The suspension was cooled and coarsely milled in liquid nitrogen. The solids were placed in a tray for menthol sublimation (13 h at 35 C 0.2 mbar, 4 h at 38 0.2 mbar). Residual menthol content in the sublimate did not exceed 0.1% w/w.

The sublimate (1.0 g) was mixed with 4.0 g lactose for inhalation (Respitose SV003, DMV) in a mixing apparatus for 1 minute. The blended powders were sieved first through 150 and then through 75μ metal sieves. The blending and sieving process was repeated. The final product contained 250 μg Fluticasone propionate in a 12.5 mg powder blend.

The particle size distribution of the active after dispersing the sample in water and dissolving the lactose (Mastersizer 2000, Malvern) was d(0.1) 0.07 μm, d(0.5) 0.16 μm and d(0.9) 1.9 μm.

The product properties were examined on NGI impactor (Cyclohaler) after the powder was packed in capsules (gelatin, size 3):
Delivered dose:196 μg
Total active passed pre-separator: 109 μg
Fine particle fraction ≦5 um: 83.1 μg

B: Study of Lung Deposition and Pharmacokinetics in Plasma

The objective of this study was to compare the relative bioavailability of a test formulation of 250 μg Fluticasone proprionate to the commercially available product Fixotide Diskus 250 μg in both the lung tissue and in the blood of beagle dogs. In both cases the drug formulation, a powder, was delivered by the inhalation route via an endotrachial tube. The new formulation was tested against the commercial product for both lung deposition and subsequent systemic absorption from the lung.

The lung deposition serves as a measure of improved delivery of this drug while the systemic absorption serves as a model of improved systemic absorption from the lung obtainable for drugs when treated with the “sublimation micronization” process. The manufacture of the improved formulation, Fluticasone Propionate on Lactose for DPI-Teva, is described above in Section A.

Test Facility: Charles River Laboratories, Tranent, Edinburgh, UK

Products studied:
1) Test—
   a) Active ingredient—Fluticasone proprionate
   b) Description—Fluticasone Propionate on Lactose for DPI-Teva, powder in glass vial
   c) Drug content—250 μg per 12.5 mg powder
   d) Batch number—MPL-80
2) Reference—
   a) Active ingredient—Fluticasone proprionate
   b) Description—Flixotide Diskus 250 mcg (GSK) (removed from blister)
   c) Drug content—250 μg per 12.5 mg powder
   d) Batch number—0806

Number of test animals: Five male beagle dogs of 4-6 months age, 6-8 kg each, per arm divided into two groups (animals 1-5 test, animals 6-10 reference).

Study Design—

| Phase | Group | Treatment | Animal no. |
|---|---|---|---|
| A | 1 | PK Blood sampling | 1-5 |
| A | 2 | PK Blood sampling | 6-10 |
| B | 1 | Lung deposition | 1-5 |
| B | 2 | Lung deposition | 6-10 |

Dosing: Inhalation dosing was carried out by intubation with an endotrachial tube under anesthesia. The formulation being tested was weighed into a pan from which the drug was dosed to the lung through a PennCentury® delivery device inserted into the endotrachial tube until the bronchi. About 12.5 mg each of the test and reference formulations were administered using an automated solenoid valve to coincide with the beginning of inspiration. In Phase A each dog was administered the formulation for its group and blood samples were taken. After a 10 day recovery/washout period the dogs were redosed in Phase B in the same manner to determine lung deposition. After each dosing, the delivery device was removed and washed with 10 ml of acetate buffer: methanol: acetonitrile (40:30:30). The wash was collected and analyzed to determine what part of the administered dose remained in the delivery device. This data was used to correct for administered dose in the pharmacokinetic calculations.

Blood sampling: Whole blood samples of 1.5 ml were collected from an appropriate vein at pre-dose, end of dose (˜5 minutes), 10, 15, 30, and 60 minutes and at 2, 4, 8 and 24 hours and transferred to lithium heparin tubes. The plasma was separated by centrifugation at 3000 rpm at about 4° C. for 15 minutes. The plasma was frozen at −80° C. until analyzed using a validated HPLC MS/MS method.

Lung sampling: The animals were euthanized 5 minutes after formulation administration in Phase B by an intravenous overdose of sodium phenobarbitone followed by severance of major blood vessels. The lungs were removed, separated into lobes, homogenized and stored frozen at −80° C. until analyzed using a validated HPLC MS/MS method.

Results:

Table 11 shows the results obtained from the analysis of fluticasone levels in the plasma of the animals receiving the test formulation by inhalation as a function of time while Table 12 shows the same data for the animals receiving the reference formulation. Table 13 presents the pharmacokinetic parameters calculated from the data in Tables 11 and 12.

TABLE 11
Plasma levels of fluticasone after inhaling test formulation

| time (hr) | test 1 | test 2 | test 3 | test 4 | test 5 |
|---|---|---|---|---|---|
| 0 | 0.000 | 0.000 | 0.000 | 0.000 | 0.000 |
| 0.025 | 0.329 | 0.364 | 0.042 | 0.159 | 0.000 |
| 0.1666 | 0.367 | 0.672 | 0.464 | 0.447 | 0.144 |
| 0.25 | 0.486 | 0.450 | 0.401 | 0.447 | 0.176 |
| 0.5 | 0.400 | 0.545 | 0.237 | 0.507 | 0.231 |
| 1 | 0.276 | 0.428 | 0.207 | 0.359 | 0.126 |
| 2 | 0.118 | 0.195 | 0.097 | 0.163 | 0.043 |
| 4 | 0.033 | 0.083 | 0.033 | 0.060 | 0.000 |
| 8 | 0.000 | 0.000 | 0.000 | 0.000 | 0.000 |
| 24 | 0.000 | 0.000 | 0.000 | 0.000 | 0.000 |

TABLE 12
Plasma levels of fluticasone after inhaling reference formulation

| Time | | | | | |
|---|---|---|---|---|---|
| (hr) | ref 6 | ref 7 | ref 8 | ref 9 | ref 10 |
| 0 | 0.000 | 0.000 | 0.000 | 0.000 | 0.000 |
| 0.025 | 0.000 | 0.000 | 0.000 | 0.000 | 0.000 |
| 0.1666 | 0.107 | 0.163 | 0.144 | 0.034 | 0.086 |
| 0.25 | 0.142 | 0.125 | 0.157 | 0.046 | 0.147 |
| 0.5 | 0.142 | 0.160 | 0.169 | 0.039 | 0.159 |
| 1 | 0.105 | 0.140 | 0.121 | 0.000 | 0.138 |
| 2 | 0.056 | 0.087 | 0.083 | 0.000 | 0.089 |
| 4 | 0.000 | 0.044 | 0.030 | 0.000 | 0.040 |
| 8 | 0.000 | 0.000 | 0.000 | 0.000 | 0.000 |
| 24 | 0.000 | 0.000 | 0.000 | 0.000 | 0.000 |

TABLE 13
Pharmacokinetic parameters calculated for test and
reference formulations
Results of fluticasone from inhaler-dogs
Average delivered dose, test (mg) = 0.190
Average delivered dose, ref (mg) = 0.140

| | AUC | | | Cmax |
|---|---|---|---|---|
| vol-sess | (h * ng/g) | t½ (h) | Tmax (h) | (ng/g) |
| 1 (test) | 0.783 | 1.0 | 0.25 | 0.486 |
| 2 (test) | 1.247 | 1.3 | 0.17 | 0.672 |
| 3 (test) | 0.610 | 1.2 | 0.17 | 0.464 |
| 4 (test) | 1.022 | 1.2 | 0.50 | 0.507 |
| 5 (test) | 0.292 | 0.7 | 0.50 | 0.231 |
| 6 (ref) | 0.251 | 1.1 | 0.25 | 0.142 |
| 7 ref | 0.467 | 1.8 | 0.17 | 0.163 |
| 8 (ref) | 0.410 | 1.5 | 0.50 | 0.169 |
| 9 (ref) | 0.026 | | 0.25 | 0.046 |
| 10 (ref) | 0.451 | 1.7 | 0.50 | 0.159 |
| AVG (test) | 0.791 | 1.1 | 0.32 | 0.472 |
| AVG (ret) | 0.321 | 1.5 | 0.33 | 0.136 |
| geomn (test) | 0.708 | 1.0 | 0.28 | 0.447 |
| geomn (ref) | 0.224 | 1.5 | 0.31 | 0.123 |
| stddev (test) | 0.369 | 0.25 | 0.17 | 0.158 |
| stddev (ref) | 0.186 | 0.32 | 0.16 | 0.051 |
| % CV (test) | 46.61% | 23.99% | 53.25% | 33.43% |
| % CV (ret) | 57.86% | 21.22% | 46.41% | 37.77% |

A comparison of Tables 11 and 12 shows very clearly that the absorption of fluticasone from the test formulation gives higher drug levels in the plasma over the entire experiment. Particularly striking is the comparison of values at the 5 minute point where the reference shows no absorbed fluticasone while the test formulation shows appreciable absorption. These results imply that the test formulation is more available in the deep lung and more soluble than the reference formulation.

The qualitative interpretations of the data in Tables 11 and 12 are borne out by the calculated pharmacokinetic parameters in Table 13. The test formulation delivered more drug from the device than did the reference formulation (190 μg vs. 140 μg). The average area under the curve (AUC) for the test formulation was more than twice that of the reference formulation (0.791 ng*h/ml vs. 0.321 ng*h/ml) and the maximum concentration (Cmax) was more than three times greater (0.472 ng/ml vs. 0.136 ng/ml).

Table 14 collects the data for fluticasone found in the various lobes of the lungs of the dogs administered the test formulation while Table 15 gives the same data for the dogs receiving the reference formulation.

TABLE 14
Fluticasone found in lung tissue of animals receiving test formulation

| | animal | animal | animal | animal | animal | |
|---|---|---|---|---|---|---|
| lobe | 1 | 2 | 3 | 4 | 5 | average |

fluticasone ng/g of lung tissue test

| | | | | | | |
|---|---|---|---|---|---|---|
| left anterior | 34.6 | 25.8 | 103.0 | 96.0 | 32.7 | 58.42 |
| left middle | 60.9 | 24.8 | 64.3 | 96.1 | 17.4 | 52.70 |
| left post | 54.1 | 77.2 | 153.0 | 139.0 | 16.5 | 87.96 |
| right | 129.0 | 90.4 | 182.0 | 148.0 | 26.9 | 115.26 |
| anterior | | | | | | |
| right middle | 63.7 | 142.0 | 220.0 | 189.0 | 27.6 | 128.46 |
| right post | 68.0 | 245.0 | 258.0 | 266.0 | 9.4 | 169.28 |
| accessory | 100.0 | 186.0 | 253.0 | 239.0 | 29.1 | 161.42 |

fluticasone total ng per lobe test

| | | | | | | |
|---|---|---|---|---|---|---|
| left anterior | 498 | 250 | 936 | 738 | 400 | 564.40 |
| left middle | 616 | 140 | 448 | 731 | 140 | 415.00 |
| left post | 2442 | 1966 | 4059 | 3273 | 551 | 2458.20 |
| right | 3464 | 1452 | 2746 | 2102 | 540 | 2060.80 |
| anterior | | | | | | |
| right middle | 987 | 1125 | 2251 | 1181 | 233 | 1155.40 |
| right post | 3138 | 5858 | 6548 | 5634 | 306 | 4296.80 |
| accessory | 1037 | 1693 | 1892 | 1489 | 259 | 1274.00 |
| total lung | 12182 | 12484 | 18880 | 15148 | 2429 | 12224.60 |

TABLE 15
Fluticasone found in lung tissue of animals receiving reference formulation

| | animal | animal | | animal | animal | |
|---|---|---|---|---|---|---|
| lobe | 6 | 7 | animal 8 | 9 | 10 | average |

fluticasone ng/g of lung tissue reference

| | | | | | | |
|---|---|---|---|---|---|---|
| left anterior | 15.6 | 47.7 | 17.4 | 39.4 | 33.3 | 30.68 |
| left middle | 22.2 | 20.4 | 17.6 | 31.0 | 37.4 | 25.72 |
| left post | 28.4 | 64.0 | 21.9 | 32.8 | 53.9 | 40.20 |
| right | 45.5 | 83.3 | 43.4 | 63.8 | 50.5 | 57.30 |
| anterior | | | | | | |
| right | 43.1 | 101.0 | 18.8 | 48.4 | 67.1 | 55.68 |
| middle | | | | | | |
| right post | 49.5 | 80.6 | 20.3 | 35.5 | 60.8 | 49.34 |
| accessory | 49.7 | 101.0 | 23.6 | 42.4 | 71.9 | 57.72 |

fluticasone total ng per lobe reference

| | | | | | | |
|---|---|---|---|---|---|---|
| left anterior | 114 | 568 | 179 | 463 | 276 | 320.00 |
| left middle | 134 | 176 | 130 | 282 | 193 | 183.00 |
| left post | 657 | 2061 | 805 | 1036 | 1335 | 1178.80 |
| right | 641 | 1863 | 1074 | 1272 | 747 | 1119.40 |
| anterior | | | | | | |
| right | 323 | 1209 | 226 | 546 | 503 | 561.40 |
| middle | | | | | | |
| right post | 1056 | 2427 | 918 | 957 | 1467 | 1365.00 |
| accessory | 314 | 957 | 254 | 444 | 468 | 487.40 |
| total lung | 3239 | 9261 | 3586 | 5000 | 4989 | 5215.00 |

The data presented in these two tables again show a distinct advantage for the test formulation over the reference formulation. In each lobe there was a two to three fold advantage of the test formulation compared to the reference formulation. Total lung deposition for the test formulation was 12 to 18 μg for 4 of the five dogs with one dog having only 2.4 μg deposited. The values for the reference formulation were 3 to 9 μg. The average value of total lung deposition for the test formulation was 12.2 μg (14.7 μg if the one low value is disregarded) while for the reference formulation the average of lung deposition was 5.2 μg. The test formulation has therefore more than twice the lung deposition of the reference formulation.

### EXAMPLE 10

### Calcitriol in Menthol with Antioxidant

Menthol, 12 grams, was melted at 50° C. and purged with a flow of nitrogen for one hour. The antioxidants butylated hydroxytoluene (267 mg) and butylated hydroxyanisole (267 mg) were added to the menthol melt. The menthol melt was stirred under nitrogen until all the antioxidants dissolved. Calcitriol (267 mg) was added to the melt which was stirred under a nitrogen atmosphere until all had dissolved. The vessel was tightly closed. The menthol solution solidified in the vessel upon cooling to room temperature (RT, ca 25° C.). The product obtained was stored in the vessel at −20 C.

### EXAMPLE 11

### Azithromycin in Menthol

Menthol (10 grams) was melted on a stirring hot plate with magnetic stirring, then heated to the desired temperature indicated in Table 1. The Azithromycin was added in small increments (0.1 grams) and stirred to obtain a clear solution. The drug was added in increments until no more drug dissolved in the menthol. The weight of material added to the menthol melt that still gave a clear solution was taken as the solubility of the active drug at the indicated temperature. The results for Azithromycin are given below.

TABLE 16

| | | Solubility |
|---|---|---|
| Active drug substance | temperature (° C.) | (% w/w) |
| Azithromycin | 63 | 40.0 |

### EXAMPLE 12

### Azithromycin on Lactose for Inhalation

The two formulations in Table 17 were prepared as follows:

Menthol was melted with stirring. Hydroxypropylcellulose LF and Azithromycin were added and the mixture stirred until all dissolved. The lactose fractions were added and stirred until a uniform suspension was obtained. The mixture was flash frozen by pouring it, along with a stream of liquid nitrogen, onto the screen of a mill so that the frozen solution was milled to small pieces (<1 mm). The menthol was sublimed from the mixture in a lyophilizer.

TABLE 17

| | |
|---|---|
| Batch 1 | Batch 2 |

| Material | gms | % | gms | % |
|---|---|---|---|---|
| Menthol | 240 | 66.7 | 240 | 64.9 |
| Azithromycin | 10 | 2.8 | 20 | 5.4 |
| HPC LF | 10 | 2.8 | 10 | 2.7 |
| Lactose micronized | 30 | 8.3 | 30 | 8.1 |
| Lactose respiratory grade | 70 | 19.4 | 70 | 18.9 |

The two batches were tested for particle size in a Malvern laser light scattering apparatus for particle size in Azithromycin saturated water such that the lactose and HPC dissolves but the Azithromycin stays in the solid state. The particles were also measured on a ‘New Generation Impactor’ (NGI) device where the total FPF were measured by HPLC on the various stage plates of the device. The NGI serves as a model for inhalation where the product is loaded into a “Cyclohaler” DPI device and tested in an airflow. The results are presented in Table 18.

TABLE 18

| | D (0.1) (μm) | D (0.5) (μm) | D (0.9) (μm) | % FPF |
|---|---|---|---|---|
| Batch 1 | 1.8 | 5.2 | 14.0 | 45.6 |
| Batch 2 | 2.0 | 6.6 | 17.3 | 36.3 |

Both batches of Azithromycin formed micrometer sized particles with 50% of the particles smaller than 5.2 or 6.6 μm respectively. The material treated with a larger ratio of menthol gave the smaller particle fraction. The results of the solution particle size determination is reflected in the solid state NGI results where Batch 1 had a larger fraction of small particles than did Batch 2.

### EXAMPLE 13

The formulation described in Table 19 is produced by the same methods as in Example 12. The amount of menthol is raised to obtain smaller particles. The calcitriol and antioxidant are added before the lactose is added. The formulation produced contains a dose of 2.5 mg azithromycin and 2 μg calcitriol for every DPI dose of 25 mg lactose.

TABLE 19

| Batch 3 | | |
|---|---|---|
| Material | Gm | % |
| Menthol | 500 | 80.6 |
| Azithromycin | 10 | 1.6 |
| HPC LF | 10 | 1.6 |
| Calcitriol | 0.008 | 0.0013 |
| BHA (antioxidant) | 0.008 | 0.0013 |
| Lactose micronized | 30 | 4.8 |
| Lactose respiratory grade | 70 | 11.3 |

The mixed active ingredient has a D(0.5) of 0.8 μm and each active ingredient separately has a >50% FPF in an NGI test where each active is separately determined by HPLC on the various stages.

1. A pharmaceutical composition comprising a micronized pharmaceutical carrier bearing micronized drug microparticles.

2. The pharmaceutical composition of claim 1, wherein the micronized pharmaceutical carrier is selected from the group consisting of lactose, dextran, dextrose, mannitol, and mixtures thereof.

3. The pharmaceutical composition of claim 1, wherein the micronized pharmaceutical carrier comprises lactose.

4. The pharmaceutical composition of claim 1, wherein the micronized pharmaceutical carrier consists essentially of lactose.

5. The pharmaceutical composition of claim 3, wherein the micronized lactose has a particle size distribution of d₅₀ less than or equal to 5 μm and d₉₀ less than or equal to 9 μm.

6. The pharmaceutical composition of claim 3, wherein the micronized lactose has a particle size distribution of d₉₀ less than or equal to 5 μm.

7. The pharmaceutical composition of claim 1, wherein the pharmaceutical composition is suitable for administration by inhalation.

8. A pharmaceutical composition comprising a pharmaceutical carrier bearing micronized drug microparticles, wherein the drug microparticles have a d₅₀ value of less than or equal to about 2 μm, wherein the composition is suitable for administration by inhalation.

9. (canceled)

10. The pharmaceutical composition of claim 1, wherein the micronized drug microparticles are non-mechanically micronized drug microparticles.

11. The pharmaceutical composition of claim 10, wherein the non-mechanically micronized drug microparticles are selected from the group consisting of docetaxel, beclomethasone, fluticasone, budesonide, salbutamol, terbutaline, ipratropium, oxitropium, formoterol, salmeterol, tobramycine and tiotropium.

12. The pharmaceutical composition of claim 10, wherein the non-mechanically micronized drug microparticles are docetaxel, beclomethasone, or fluticasone.

13. (canceled)

14. The pharmaceutical composition of claim 1, further comprising a non-micronized pharmaceutical carrier.

15.-21. (canceled)

22. The pharmaceutical composition of claim 1, wherein the pharmaceutical composition is suitable for administration by dry powder inhalation.

23. A method of preparing a pharmaceutical composition comprising the steps of:
a) providing a solid solution of a drug and a sublimable carrier on the surface of a pharmaceutical carrier particle, and
b) subliming the sublimable carrier from the solid solution, thereby depositing micronized microparticles of the drug on the surface of the pharmaceutical carrier particle to obtain a pharmaceutical carrier bearing micronized drug microparticles, wherein the drug microparticles have a d₅₀ value of less than or equal to about 2 μm.

24.-25. (canceled)

26. A pharmaceutical composition for administration by injection comprising a pharmaceutical carrier suitable for reconstitution into an injectable solution or suspension bearing non-mechanically micronized drug microparticles having a d₅₀ value of less than 2 μm.

27.-33. (canceled)

34. The pharmaceutical composition of claim 1, wherein the micronized drug microparticles are deposited on the pharmaceutical carrier from a solid solution of the drug in a sublimable carrier.

35. A method of making a pharmaceutical composition comprising the steps of:
a) providing a solid solution of a drug and a sublimable carrier on the surface of a micronized pharmaceutical carrier particle, and
b) subliming the sublimable carrier from the solid solution, thereby depositing micronized microparticles of the drug on the surface of the micronized pharmaceutical carrier particle.

36.-50. (canceled)

51. The method of claim 35, wherein step a) comprises:
applying a combination of the drug and molten sublimable carrier to the surface of at least one micronized pharmaceutical carrier particle, and solidifying the combination by flash freezing to obtain the solid solution.

52.-53. (canceled)

54. A pharmaceutical composition prepared by a process comprising the steps of:
a) providing a solid solution of a drug and a sublimable carrier on the surface of a micronized pharmaceutical carrier particle, and
b) subliming the sublimable carrier from the solid solution, thereby depositing micronized microparticles of the drug on the surface of the micronized pharmaceutical carrier particle.

55.-56. (canceled)

57. The pharmaceutical composition of claim 54, wherein step a) in the process comprises:
applying a combination of the drug and molten sublimable carrier to the surface of at least one pharmaceutical carrier particle, and solidifying the combination by flash freezing to obtain the solid solution.

58.-60. (canceled)

61. A method of treatment comprising administering by inhalation the pharmaceutical composition of claim 1.

62. A method of treatment comprising administering by injection the pharmaceutical composition of claim 1.

63. A method of increasing the plasma level of a drug in a patient comprising administering a pharmaceutical composition of claim 1, and containing said drug, to a patient in need of an increased plasma level of said drug.

64. A composition suitable for pulmonary delivery comprising microparticles of a vitamin D compound and particles of a pharmaceutically acceptable carrier.

65.-77. (canceled)

78. A method for preparing a pharmaceutical composition comprising:
a) providing a solid solution of a vitamin D compound, a pharmaceutically acceptable carrier, and a sublimable carrier; and
b) subliming the sublimable carrier from the solid solution to form the pharmaceutical composition.

79.-83. (canceled)

84. A method of treating lung infection associated with cystic fibrosis comprising delivering calcitriol to the lung by inhalation.

85.-89. (canceled)

90. A method of preparing calcitriol for pulmonary delivery comprising:
a) dissolving calcitriol in a sublimable solvent to form a solution;
b) mixing the solution with a pharmaceutically acceptable carrier;
c) optionally adding at least one pharmaceutical additive to the solution;
d) solidifying the solution to solid solution on the carrier; and
e) subliming the sublimable solvent.

91.-96. (canceled)

97. A method of treating lung infection in a patient with cystic fibrosis comprising delivering an antibiotic to the lung by inhalation, wherein the antibiotic is in particle form and the particles have a diameter of less than about 3000 nm.

98.-104. (canceled)

105. A composition suitable for pulmonary delivery comprising azithromycin, wherein the azithromycin is in particle form and the particles have a diameter of less than about 3000 nm.

106.-114. (canceled)

115. A method for preparing azithromycin for pulmonary delivery comprising:
a) dissolving azithromycin in a sublimable solvent to form a solution;
b) mixing the solution with a carrier;
c) optionally adding at least one additional pharmaceutical additive;
d) solidifying the solution to a solid solution on the carrier; and
e) subliming the sublimable solvent.

116.-119. (canceled)

120. A composition comprising azithromycin, wherein the azithromycin is in particle form and the particles have a diameter of less than about 3000 nm.

121. (canceled)

122. A composition comprising calcitriol, wherein the calcitriol is in particle form and the particles have a diameter of less than about 3000 nm.

123. (canceled)

124. The composition of claim 120, wherein the composition further comprises calcitriol particles having a diameter of less than about 3000 nm.

125.-127. (canceled)
